(19) United States
(12) Patent Application Publication
(10) Pub. No.: US 2002/0110835 A1
(43) Pub. Date: Aug. 15, 2002

(54) Microfluidic devices and methods

(76) Inventor: Rajan Kumar, Robbinsville, NJ (US)

Correspondence Address: Rajan Kumar, 18 Buford Road, Robbinsville, NJ, 08691, US

(21) Appl. No.: 10/076,741

(22) Filed: Feb. 13, 2002

Related U.S. Application Data
(60) Provisional application No. 60/268,132, filed on Feb. 13, 2001.

(51) Int. Cl. C12Q 1/68; G01N 33/53; C12M 1/34

(52) U.S. Cl. 435/7.1; 435/287.2; 438/1; 435/6

(57) ABSTRACT

This invention describes novel microfluidic methods for performing biochemical analysis involving multiple steps. Microfluidic devices that can be used for performing such assays are also described. The uses of such methods include genomic and proteomic analysis, diagnostic assays, and drug discovery.

### CROSS-REFERENCE TO RELATED APPLICATIONS

This application claims the benefit under 35 U.S.C. 119(e) from U.S. Provisional Application No. 60/268,132, filed 2/13/2001.

This application is related to U.S. Non-Provisional Application No. 10/052,452, filed 10/25/2002, U.S. Non-Provisional Application No. 10/1017,536, filed 10/29/2002, and U.S. Non-Provisional Application No. 10/010,684, filed 12/05/2001. These applications are incorporated herein by reference.

### STATEMENT REGARDING FEDERALLY SPONSORED RESEARCH OR DEVELOPMENT

The invention was funded in part by the U.S. Government through a Department of Energy grant (DE-FG02-01ER83195).

### BACKGROUND OF THE INVENTION

The present invention lies in the field of molecular biology and is particularly concerned with the use of fluidic devices for detection of molecules of interest in a sample, determination of composition of a complex mixture of molecules, such molecules including although not exclusively, DNA, RNA and proteins, and performing biochemical processes including sample preparation.

Sequencing of a large number of genomes has generated a growing body of DNA sequence information that promises to revolutionize experimental design and data interpretation in pursuit of biological understanding. However, collection of sequence data, by itself, is not sufficient to decipher the roles of genes and gene products in cellular and organismal function. Therefore, there has been a concomitant growth in development of technologies to exploit the massive amount of DNA sequence data.

One of such revolutionary technologies in the biotechnology area is the emergence of fluidic biochips for performing genomics, diagnostics and pharmaceutical assays. Assays based on the microfluidic technology promise to enhance the throughput of biochemical and pharmaceutical analysis, and provide significant cost advantages by reducing reagent consumption. Typically, microfluidic assays are conducted in glass or plastic devices with channels in the order of 10-1000 micron width and height. The reagents for the assays are added to the channels and allowed to react. The output of the reaction is measured by a detectable change in the reactants.

A number of biologically relevant processes have been adapted to fluidic chips that have demonstrated better characteristics including higher kinetics and reduced reagent consumption. High throughput assays can be performed in fluidic chips with lower cost of reagents, and lower cost of manufacturing due to mass production of chips using IC/MEMS manufacturing techniques. In addition, fluidic chips enable design of novel chemistries for improved assay performance.

However, despite all the potential, fluidic chips have been limited in their use over the last decade. We believe that this is due to the following reasons:

Most biological assays are heterogeneous assays, i.e. consist of reagents in liquid phase as well as solid phase. The solid phase is usually used to capture products of a reaction for further processing or analysis. In fluidic devices, the capture ability is introduced by covalently attaching capture reagents in relevant areas of the chips, e.g. oligonucleotide probes for detection of specific DNA in a sample. However, such functionalization of chips makes them non-reusable. Non-reusability of chips has limited design and applications of complex fluidic chips.

Complex fluidic chips are expensive to fabricate and are not suitable for disposable applications. Therefore, in the absence of an ability to reuse chips, complex fluidic chips are not practical and commercially viable.

Attachment of specific molecules in the chip makes the manufacturing process highly customized for each chip. In addition to introducing complexity to the manufacturing process, this results in loss of cost advantage due to mass production. Thus mass production of generic chips is desirable followed by easy, quick and cost effective customization. In the absence of such customization after chip fabrication, the cost and mass-production benefits of microfluidic chips cannot be realized. A number of methods have been investigated to customize chips after their fabrication. Beads, usually superparmagnetic beads, that are functionalized with desired properties have been introduced into the chip. The beads are separated from the liquid for analysis. Different methods for separation of beads have been proposed. However, due to tendency of beads to aggregate and clog the channels, this alternative is not practical for commercial systems.

In order to realize the full potential of biochips, a number of challenges must be met. There is a need to develop the ability to re-use fluidic chips, and to mass-produce generic chips at a low cost. In addition, there is a need to develop methods that allow rapid incorporation of assays into microfluidic devices. Current methods do not allow performance of multistep protocols in microfluidic devices. Therefore, there is a need for microfluidics devices and methods that will allow easy performance of multistep protocols in microfluidics devices.

One of the major limitations of microfluidic assays is difficulty in concentrating a reactant or separating the product from the reactant. This is important when the assay being used is a multistep process with the products produced in one step being used for reactions in the next step. To achieve this goal, solid phase components are used in microfluidic devices. Most common is the use of micro-particles such as beads, which have been functionalized with a specific affinity for the desired or undesired products. By holding the beads stationary while moving the fluids separation or concentration of the captured product can be achieved. However, the handling of the beads in microfluidic devices is very difficult and usually results in clogging. It also limits the use of microfluidic devices to one assay without extensive cleaning. These limitations have prevented the development of a robust microfluidic system for biochemical analysis.

It is, therefore, an object of the present invention to provide improved fluidic methods and devices for performing microscale biochemical assays.

### BRIEF SUMMARY OF THE INVENTION

The present invention describes a novel approach to perform fluidic assays in which solid substrates carry reaction materials between different steps of a process, each of the steps being carried in a different microfluidic device. The process of moving materials consists of removing the substrate from one chip and inserting it into another chip. The process of removal and insertion can involve more than two chips used sequentially.

In another embodiment of the invention, two different substrates removed from two different chips can be inserted into a third chip.

In one embodiment, the microfluidic chips used contain one chamber for each substrate. In another embodiment, multiple substrates can be inserted into the same chamber in a chip.

It is yet another object of the present invention to describe methods to use microfluidic chips and substrates to perform complex biochemical protocols.

It is another object of the present invention to provide methods to isolate specific molecules from a sample for further processing by using substrates coated with affinity molecules.

### BRIEF DESCRIPTION OF THE SEVERAL VIEWS OF THE DRAWING

FIG. 1 is a view of one embodiment of the invention in which six substrates 21-26 are inserted into a microfluidic chip 10 containing a single chamber 12.

FIG. 2 shows a multi-layered chip composed of ten microfluidic chips (10A to 10J). Each chip contains its own chamber (e.g. 12A for chip 10A).

FIG. 3 shows a three-layered microfluidic chip in which each substrate is introduced into a separate fluidic chamber. Each layer of the chip can be used to expose the substrate to identical or different reagent. Similarly, within a layer, different reagents can be used in each chamber.

FIG. 4 shows another embodiment of the invention in which microfluidic chips containing chambers for multiple substrates and those containing chambers for single substrates are combined.

FIG. 5 shows an example protocol to perform a genetic bit analysis. FIG. 5A shows four identical assemblies of twenty substrates 47 each. FIG. 5B shows the four assemblies inserted into a twenty-chamber chip. FIG. 5C shows the same four assemblies inserted into a four-chamber chip. FIG. 5D shows the assemblies separated from the chip for detection.

### DETAILED DESCRIPTION OF THE INVENTION

Before providing a detailed description of the inventions of this patent, particular terms used in the patent will be defined.

A “microfluidic chip” is a device comprising a substrate that contains at least one channel and at least one opening that connects the channel to the outside.

A “substrate” is a solid material that can be inserted into and removed from a microfluidic chip. A substrate can contain on its surface distinct spots or deposits of one or more than one molecular species or surface modifications. An example of such molecular array is an array of antibodies specific against molecules of interest in the sample.

A “chamber” is the open space present in a microfluidic chip. A “channel” also denotes the same space, if the space is long in one dimension than the others.

“Hybridization” is the process by which two strands of DNA or RNA come together to form a double-stranded molecule. For hybridization between two strands to take place, the sequence of the two strands must be completely or nearly so complementary.

“Complementary” strand of a given strand is a strand of DNA or RNA that is able to hybridize to the given strand and is characterized by the presence of nucleotides A, C, G, and T, respectively opposite to nucleotides T, G, C, and A, respectively, on the given strand.

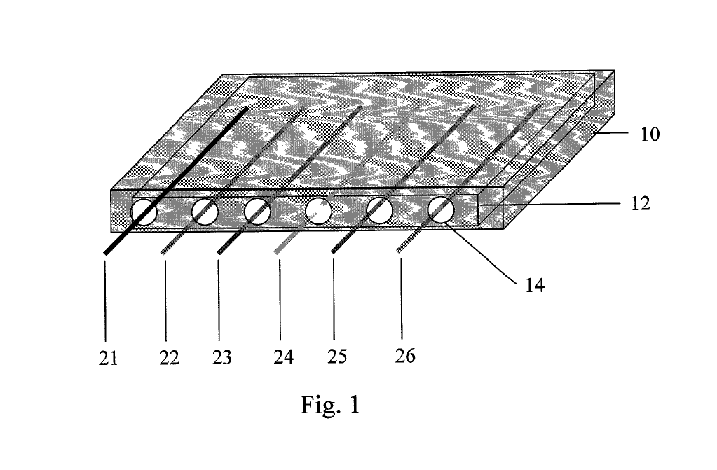

The microfluidic chips of the present invention are described with reference to FIG. 1. A microfluidic chip 10 comprising a single chamber 12 is shown. Six substrates (21, 22, . . . , 26) are inserted into the chamber through holes 14. The hole is plugged to prevent leaking. The chip of FIG. 1 can be used to expose six substrates to the same reagent.

The length and width of the chip can be between 1 and 200 millimeters, preferably between five and fifty millimeters. The height of the chip can vary between 100 microns and 10 millimeters, preferably between 500 microns and 2 millimeters. The number of substrates introduced into the chip can also vary from one to hundred and will be determined by the particular application for which the chip is designed. The size of the port for introduction of substrate will depend on the size of the substrate used, but will typically be 100 microns to 5 millimeters.

The substrates will typically be composed to glass or plastics. The seal between the substrate and the chip is composed of a plastic material such as silicone. Typical dimensions of the substrates will be 10 micron to 2 millimeters cross-sectional diameter or each side in case of non-circular substrates. Substrates will usually contain deposition of molecules such as oligonucleotides, proteins, antibodies or aptamers, which have specific affinity to molecules of interest. Alternatively, the surface of the substrates can contain modifications to allow them to capture classes of material from samples. Once substrates are removed from the microfluidic chips, they can also be interfaced with other non-microfluidic analytical instruments. For example, a substrate containing depositions of antibodies can be exposed to a sample containing proteins and allowed to capture specific proteins. Subsequently, the substrate can be interfaced with a MALDI-ToF instrument that allows identification of the proteins captured on the substrate.

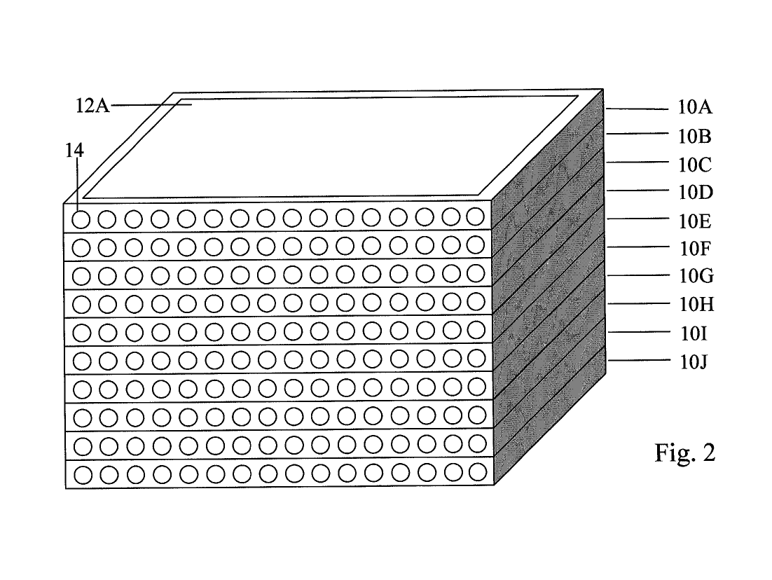

FIG. 2 shows an alternative embodiment comprising multiple chips 10A, 10B . . . ,10J stacked together. Each of the chips has its own chamber. The chamber 12A corresponding to chip 10A is visible. The substrates can be introduced into the chips from the ports 14.

In an alternative embodiment, the chip shown in FIG. 2 can have a single chamber. Alternatively, two or more chambers can be present such that each of the chambers is shared between two or more chips.

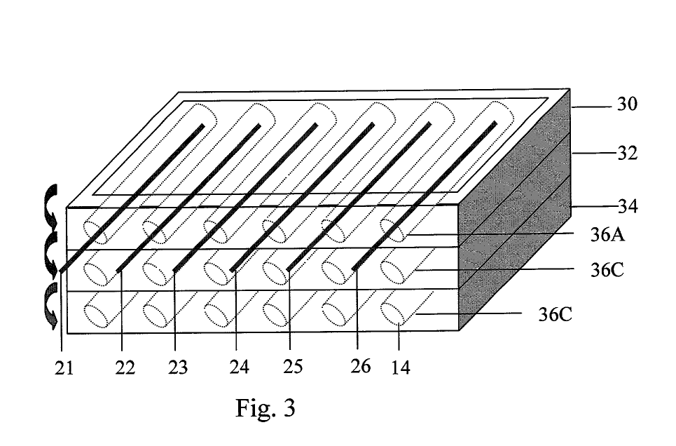

FIG. 3 shows a three-layered chip comprising chips 30, 32, and 34. Each of the comprising chips contains six individual chambers or channels 36A, 36B and 36C. These chips are configured so that substrates 21-26 are inserted into separate channels of the microfluidic chip through the ports 14 and can be exposed to different reagents in each of the channels. The arrows on the left indicate the movement of substrates between chip 30 and chip 32, and between chip 32 and chip 34.

The chip shown in FIG. 3 can be used to perform a three-step protocol. For example, the substrates can be exposed to the sample in chip 30 and allowed to capture molecules of interest. Each substrate comes in contact with a different sample in its respective channel. Subsequently, the substrates are removed from chip 30 and inserted into chip 32, where the substrates can be exposed to detection reagents that bind to the molecule captured from the sample. For example, the antibodies specific for molecules of interest can be introduced. Finally, the substrates are removed from chip 32 and inserted into chip 34, where the binding of the detection molecules on the substrates can be measured.

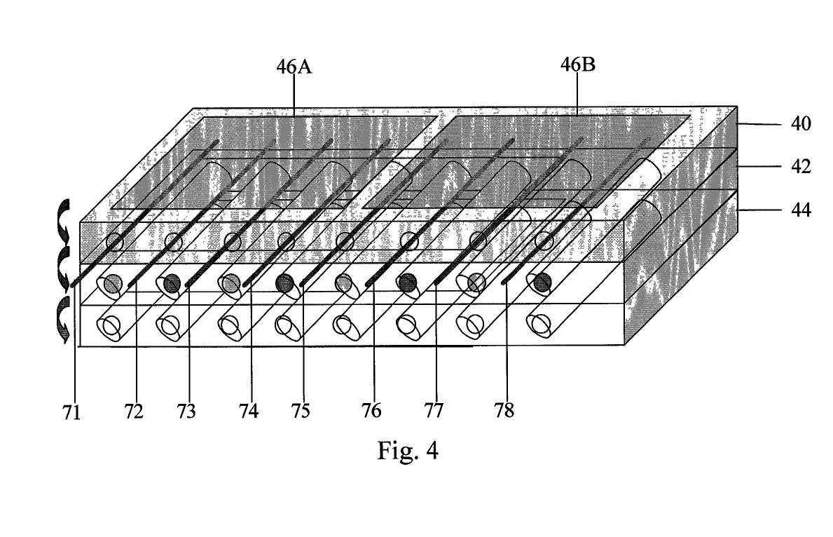

FIG. 4 shows a three-layered chip comprising different kinds of chips. Chip 40 contains two chambers 46A and 46B. Four substrates can be inserted into each of the chambers. Therefore, substrates 71, 72, 73, and 74 can be inserted into chamber 46A and substrates 75, 76, 77 and 78 are inserted into chamber 46B. Subsequently, the substrates can be removed from chip 40 and inserted into chip 42 which contains eight different chambers such that each substrate is inserted into a separate chamber. Similarly, chip 43 contains eight different chambers, one for each of the eight substrates.

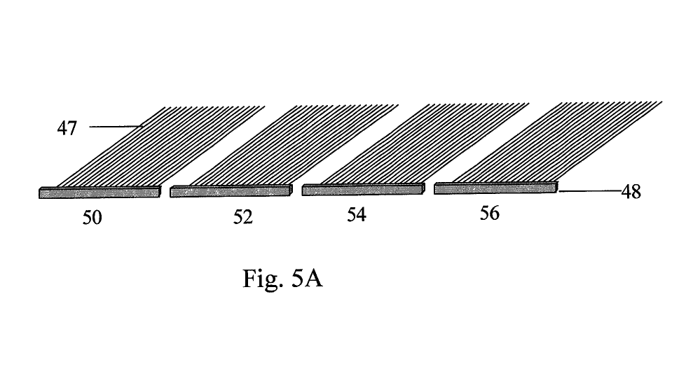

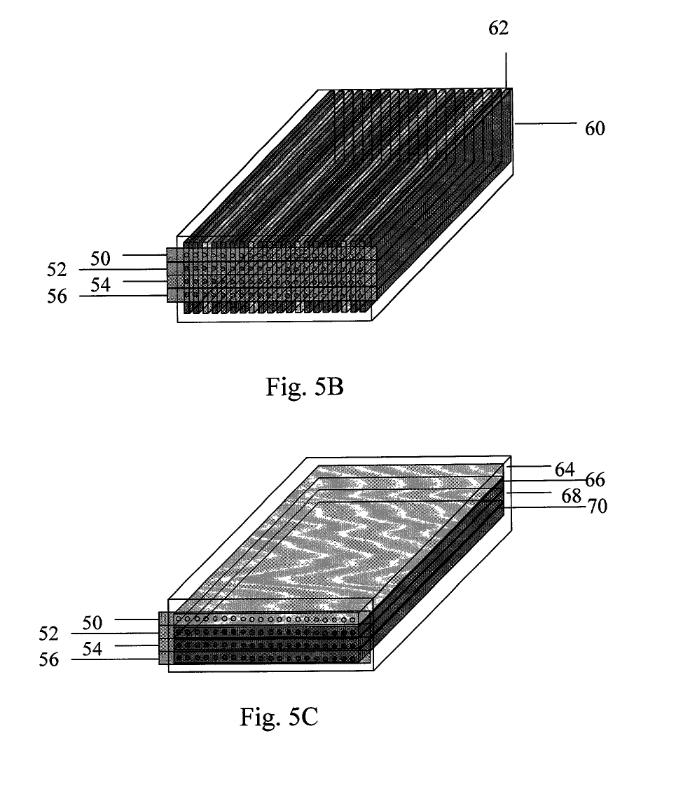

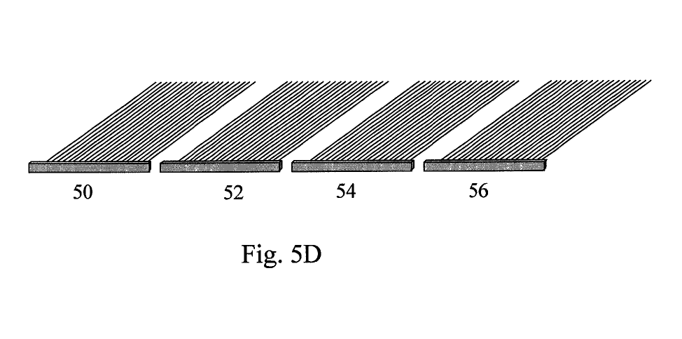

FIG. 5 shows how the movement of substrates can be used to perform a complex protocol such as Genetic Bit Analysis (GBA). GBA is used to determine the sequence of a DNA molecule at a particular location by single base-pair extension. In GBA, arrays of immobilized oligonucleotides are allowed to capture complementary DNA molecules by the process of hybridization. Subsequently, the oligonucleotides that have captured a complementary DNA molecule are extended by one nucleotide using a template-dependent DNA polymerase in the presence of one of the four nucleotides. The nucleotide added is fluorescent so that the addition of the nucleotide makes the immobilized oligonucleotide fluorescent. By determining which nucleotide is successfully added, the base-pair on the target DNA molecule can be identified.

To perform GBA in microfluidic chips according to the present invention, arrays of oligonucleotides are generated as shown in FIG. 5A. Four identical assemblies 50, 52, 54 and 56 of twenty substrates 47 are shown. Each substrate within an assembly contains multiple distinct depositions of oligonucleotides that will be used to capture the target molecules. The substrates consist of glass rods or glass tubes.

FIG. 5B shows the substrates inserted into a microfluidic chip 60. The microfluidic chip contains twenty chambers 62. One of the twenty substrates from each assembly 50, 52, 54, 56 is inserted into one of the chambers. Each chamber is then filled with a different sample and the hybridization reaction is allowed to proceed in which the target molecules present in the samples bind to depositions of oligonucleotides on the substrates. After an appropriate incubation period, the substrates are removed from the microfluidic chips.

FIG. 5C shows the next step. The chip used for this step consists of four chambers 64, 66, 68, and 70 which are oriented horizontally. The assemblies 50, 52, 54, 56 are inserted into this chip such that all substrates within an assembly are inserted into the same chamber of the chip. A different extension mix is added to each of the four chambers. Chamber 64 gets adenosine extension mix, chamber 66 gets cytosine extension mix, chamber 68 gets guanine extension mix and chamber 70 gets thymidine extension mix. During the incubation, the oligonucleotide gets extended if the target molecule contains appropriate nucleotide at its next place. For example, if a substrate shows addition of a cytosine at a particular location, it means that the target molecule contains a guanine as the next residue at that location.

FIG. 5D shows that the assemblies can be separated and studied with fluorescence detection using standard microarray scanners.

The linear depositions of functionalization can be made on the substrate using any of a number of methods. The functionalization can be performed by drawing using rollers, pens or quills or by printing using inkjet or bubble jet printers. Additionally for polymeric biological molecules such as DNA, proteins and RNA, the appropriate functionalization can be added to the substrate using in situ synthesis using photolithography or ink jet printing.

The molecules that are deposited on the substrates are usually covalently coupled to the substrate material. The choice of a particular method for coupling specific molecules to a substrate depends on characteristics of the molecules and the substrate. For example, a number of methods are known in the art for coupling DNA molecules to glass substrates, including coupling of amino-terminated nucleotides to aldehyde coated glass substrates. Similarly, a number of methods for coupling protein molecules to plastic substrates are known in the art, and can be used to create the arrays of the present invention.

Any chemistry that has been described in microfluidics and uses beads can be modified to work with substrates and microfluidic devices of the present invention. Examples of such technologies include Genetic Bit Analysis, scintillation proximity assay, etc.

The arrays of the invention can also be combined with molecular biology reagents and instructions to design kits for genomic and proteomic research as well as for drug discovery.

Methods to fabricate microfluidics chips are well-known in the art and have been described in detail in literature.

Although the invention has been described in some detail by way of illustration and example for purposes of clarity and understanding, it may be readily apparent to those of ordinary skill in the art in light of the teachings of this invention that certain changes and modifications may be made without departing from the spirit or scope of the appended claims.

Example 1

### CAPTURE AND ANALYSIS OF PROTEINS

Fabricate a microfluidic chip containing a single channel from polycarbonate sheet. Make the channel diameter 500 microns. Have the channel communicate with outside through two fluidic ports, in addition to a port for introduction of the substrate. Take a square glass tube 250 micron on each side. Make depositions of anti-TNF-alpha antibody on the glass tube. Insert the glass tube substrate into the channel in the microfluidic tube. Flow a sample solution containing TNF-alpha through the microfluidic device. After 15 minutes of the flow, stop the flow and remove the substrate. Insert the substrate into a channel on another chip and flow second antibody that is fluorescently labeled over it. After 15 minutes, remove the array and image using a fluorescence microscope. The presence of fluorescence indicates presence of TNF-alpha in the sample.

Alternatively, after exposure to sample containing TNF-alpha, the substrate can be prepared for and introduced into a mass spectrometer to detect the presence of bound TNF-alpha.

What I claim as my invention is

1. Any method for analysis of biomolecules comprising removal of a substrate from one microfluidic chip and insertion of the said substrate into a second microfluidic chip for further processing or analysis.

2. A method of claim 1 comprising the substrate is inserted into and removed from more than two chips.

3. A method of claim 2 comprising two substrates removed from two different microfluidic chips are combined for further processing in a third microfluidic chip.

4. Any microfluidic chip used for performing an analysis method of claim 1.

5. Any combination of microfluidic chips used for performing an analysis method of claim 1.

6. Any microfluidic chip of claim 3 comprising a chamber in which multiple substrates can be exposed to the same sample.

7. Any substrate used to transfer material among different microfluidic chips.

8. A substrate of claim 7 comprising an array of affinity molecules on its surface.

9. A substrate of claim 7 comprising the said substrate is composed of porous material.

10. A method of claim 1 comprising the method is used for capture and isolation of molecules of interest from the sample flowing in the microfluidic device.
